(19) United States
(12) Patent Application Publication
(10) Pub. No.: US 2025/0120678 A1
(43) Pub. Date: Apr. 17, 2025

(54) METHOD AND SYSTEM FOR AUTOMATIC REGION OF INTEREST BOX PLACEMENT AND IMAGING SETTINGS SELECTION DURING ULTRASOUND IMAGING

(71) Applicant: GE Precision Healthcare LLC, Waukesha, WI (US)

(72) Inventors: Yelena Tsymbalenko, Mequon, WI (US); Doron Shaked, Tivon (IL); Eran Shachar, Haifa (IL)

(21) Appl. No.: 18/379,269

(22) Filed: Oct. 12, 2023

(51) Int. Cl. A61B 8/00; A61B 8/08; G06V 10/12; G06V 10/22; G06V 10/25; G06V 10/764; G06V 20/50

(52) U.S. Cl. CPC A61B 8/54; A61B 8/085; A61B 8/4245; A61B 8/461; A61B 8/5207; G06V 10/12; G06V 10/225; G06V 10/25; G06V 10/764; G06V 20/50; G06V 2201/031

(57) ABSTRACT

A system and method for automatically selecting imaging settings and automatically placing a region of interest box on a first mode ultrasound image based on first mode ultrasound image information when entering a second ultrasound imaging mode is provided. The method includes acquiring first ultrasound image information, including a first mode ultrasound image, according to a first mode. The method includes processing the first mode ultrasound image to determine first mode information. The method includes automatically selecting a size and a location of a region of interest box based on the first mode information. The method includes acquiring second ultrasound image information according to a second mode based on the region of interest box. The method includes presenting, at a display system, the second ultrasound image information and the region of interest box automatically placed on the first mode ultrasound image.

### FIELD

Certain embodiments relate to ultrasound imaging. More specifically, certain embodiments relate to a method and system for automatically selecting imaging settings and automatically placing a region of interest box on a first mode ultrasound image (e.g., B-mode image) based on first mode ultrasound image information when entering a second ultrasound imaging mode (e.g., Color Flow, Power Doppler, B-Flow Color, or the like).

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

Standard ultrasound imaging views of an abdomen typically include multiple patient anatomical structures, such as a liver, kidney, gallbladder, aorta, pancreas, spleen, and/or inferior vena cava, for example. If an ultrasound operator initiates a Color Flow mode, Power Doppler mode, B-Flow Color mode, or the like, a region of interest box may be automatically positioned at a center of a B-mode image. However, the target anatomical structure may not always be located at the center of the image plane for a particular abdominal standard view. Moreover, different imaging settings may be needed to optimize the acquisition of Color Flow mode, Power Doppler mode, B-Flow Color mode, or the like ultrasound image data for different anatomical structures due to different hemodynamics of the different anatomical structures. Accordingly, current processes for positioning a region of interest box and selecting imaging settings for acquiring Color Flow mode, Power Doppler mode, B-Flow Color mode, or the like ultrasound image data for different anatomical structures is inefficient and may be difficult for less experienced ultrasound operators.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for automatically selecting imaging settings and automatically placing a region of interest box on a first mode ultrasound image based on first mode ultrasound image information when entering a second ultrasound imaging mode, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to automatically select imaging settings and automatically place a region of interest box on a first mode ultrasound image based on first mode ultrasound image information when entering a second ultrasound imaging mode, in accordance with various embodiments.

FIG. 2 illustrates a screenshot of an exemplary first mode ultrasound image having identified anatomical structures, in accordance with various embodiments.

FIG. 3 illustrates a screenshot of exemplary second ultrasound image information within a region of interest box automatically placed on a first mode ultrasound image, in accordance with various embodiments.

FIG. 4 is a flow chart illustrating exemplary steps that may be utilized for automatically selecting imaging settings and automatically placing a region of interest box on a first mode ultrasound image based on first mode ultrasound image information when entering a second ultrasound imaging mode, in accordance with various embodiments.

### DETAILED DESCRIPTION

Certain embodiments may be found in a method and system for automatically selecting imaging settings and automatically placing a region of interest box on a first mode ultrasound image (e.g., B-mode image) based on first mode ultrasound image information when entering a second ultrasound imaging mode (e.g., Color Flow, Power Doppler, B-Flow Color, or the like). For example, aspects of the present disclosure have the technical effect of automatically sizing and placing a region of interest box at a center of a target anatomical structure in a first mode ultrasound image based on analysis of first mode ultrasound image information to determine the target anatomical structure and the location of the target anatomical structure. Moreover, aspects of the present disclosure have the technical effect of automatically selecting imaging settings specific to a target anatomical structure for ultrasound image acquisition according to a second mode based on analysis of first mode ultrasound image information to determine the target anatomical structure and the location of the target anatomical structure. Furthermore, aspects of the present disclosure have the technical effect of updating a region of interest box size (i.e., geometry), region of interest box location, and/or imaging settings in response to modification of the target anatomical structure by selection of a new target anatomical structure in a standard ultrasound image view or by repositioning an ultrasound probe to acquire a new standard ultrasound image view.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode, which can be one-dimensional (1D), two-dimensional (2D), three-dimensional (3D), or four-dimensional (4D), and comprising Brightness mode (B-mode or 2D mode), Motion mode (M-mode), Color Motion mode (CM-mode), Color Flow mode (CF-mode), Pulsed Wave (PW) Doppler, Continuous Wave (CW) Doppler, Contrast Enhanced Ultrasound (CEUS), and/or sub-modes of B-mode and/or CF-mode such as Harmonic Imaging, Shear Wave Elasticity Imaging (SWEI), Strain Elastography, Tissue Velocity Imaging (TVI), Power Doppler Imaging (PDI), B-Flow Color (BFC), Micro Vascular Imaging (MVI), Ultrasound-Guided Attenuation Parameter (UGAP), and the like.

Furthermore, the term processor or processing unit, as used herein, refers to any type of processing unit that can carry out the required calculations needed for the various embodiments, such as single or multi-core Central Processing Unit (CPU), Accelerated Processing Unit (APU), Graphic Processing Unit (GPU), Digital Signal Processor (DSP), Field Programmable Gate Array (FPGA), System on a Chip (SoC), Application-Specific Integrated Circuit (ASIC), or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

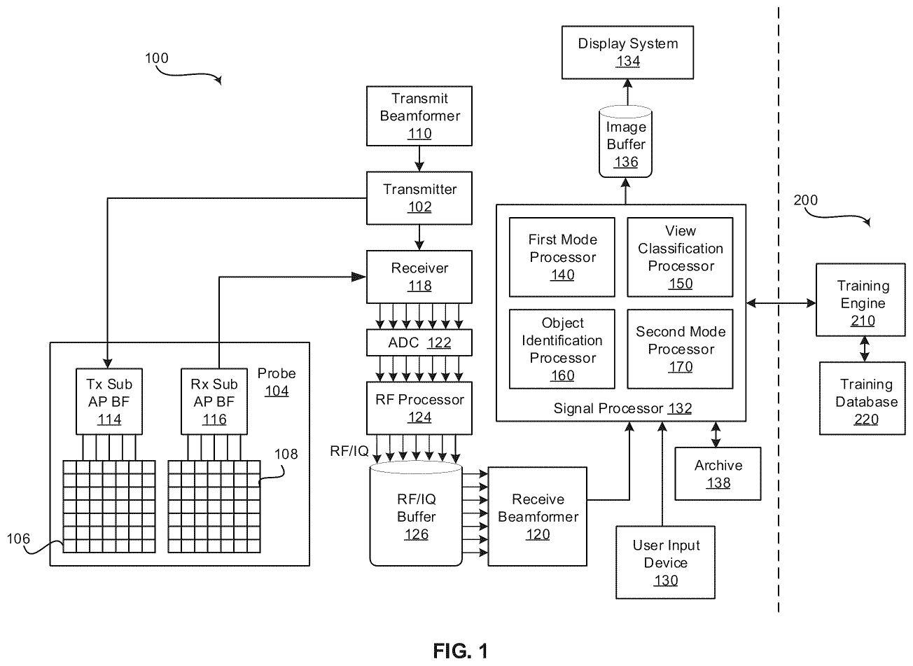

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to automatically select imaging settings and automatically place a region of interest box on a first mode ultrasound image based on first mode ultrasound image information when entering a second ultrasound imaging mode, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two-dimensional (2D) array of piezoelectric elements. In various embodiments, the ultrasound probe 104 may be a matrix array transducer or any suitable transducer operable to acquire 2D and/or 3D (including 4D) ultrasound image datasets. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as an abdomen, a heart, a fetus, a lung, a blood vessel, or any suitable anatomical structure(s).

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or a plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, image acquisition and scan parameters, settings, configuration parameters, select protocols and/or templates, change scan mode, select anatomical structure targets, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, graphic processing units, microprocessors, microcontrollers, and/or the like. The signal processor 132 may be an integrated component, or may be distributed across various locations, for example. In an exemplary embodiment, the signal processor 132 may comprise a first mode processor 140, a view classification processor 150, an object identification processor 160, and a second mode processor 170 that may be capable of receiving input information from a user input device 130 and/or archive 138, generating an output displayable by a display system 134, and manipulating the output in response to input information from a user input device 130, among other things. The signal processor 132, first mode processor 140, view classification processor 150, object identification processor 160, and second mode processor 170 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a first mode processor 140 that comprises suitable logic, circuitry, interfaces and/or code that may be operable to process an acquired and/or retrieved first mode ultrasound image dataset to generate ultrasound images according to a first mode. As an example, the first mode may be a 2D mode (e.g., B-mode, biplane mode, triplane mode, or the like) and the first mode processor 140 may be configured to process a received first mode ultrasound image dataset into 2D image(s). The first mode image(s) may be provided to the view classification processor 150, provided to the object identification processor 160, presented at the display system 134 and/or stored at archive 138 or any suitable data storage medium.

The signal processor 132 may include a view classification processor 150 that comprises suitable logic, circuitry, interfaces and/or code that may be operable to process the first mode ultrasound image generated by the first mode processor 140 to determine a standard view depicted in the first mode ultrasound image. For example, the view classification processor 150 may determine which of a plurality of abdominal standard views the first mode ultrasound image is representing. In various embodiments, the standard view is associated with a target anatomical structure. Accordingly, determining the standard view by the view classification processor 150 identifies a target anatomical structure. In an exemplary embodiment, the processing of the first mode ultrasound image by the view classification processor 150 may be initiated in response to receiving a user selection to switch to a second mode, such as Color Flow, Power Doppler, B-Flow Color, or the like. In certain embodiments, the view classification processor 150 may continuously process received first mode ultrasound images to detect if the ultrasound probe 104 was moved to acquire a different standard view. Alternatively, after initial processing of the first mode ultrasound images, subsequent processing of the first mode ultrasound images may be triggered by a detected change to the target anatomical structure, as described in more detail below.

The view classification processor 150 may include image analysis algorithms, artificial intelligence algorithms, one or more deep neural networks (e.g., a convolutional neural network) and/or may utilize any suitable form of image analysis techniques or machine learning processing functionality configured to automatically determine a standard view depicted in a first mode ultrasound image, such as an abdomen standard view depicted in a B-mode image. In various embodiments, the view classification processor 150 may be provided as a deep neural network that may be made up of, for example, an input layer, an output layer, and one or more hidden layers in between the input and output layers. Each of the layers may be made up of a plurality of processing nodes that may be referred to as neurons. For example, the view classification processor 150 may include an input layer having a neuron for each pixel or group of pixels from the ultrasound image data. The output layer may have neurons corresponding to a classification of the standard view depicted in the ultrasound image data. Each neuron of each layer may perform a processing function and pass the processed ultrasound image information to one of a plurality of neurons of a downstream layer for further processing. The processing performed by the view classification processor 150 deep neural network (e.g., convolutional neural network) may classify standard views depicted in ultrasound image data with a high degree of probability. The view classification processor 150 may provide the classification of the standard view depicted in the first mode ultrasound image to the object identification processor 160, provide the standard view classification to the second mode processor 170, present the standard view classifications at the display system 134 and/or may store the standard view classification at archive 138 and/or any suitable data storage medium.

The signal processor 132 may include an object identification processor 160 that comprises suitable logic, circuitry, interfaces and/or code that may be operable to process the first mode ultrasound image generated by the first mode processor 140 to determine anatomical structure locations depicted in the first mode ultrasound image. For example, the object identification processor 160 may determine locations of one or more of a liver, kidney, gallbladder, aorta, pancreas, spleen, and/or inferior vena cava, among other anatomical structures, depicted in a first mode ultrasound image that is an abdominal standard view. In various embodiments, the object identification processor 160 may receive a standard view classification from the view classification processor 150. The standard view identified by the view classification processor 150 is associated with a target anatomical structure. Accordingly, the object identification processor 160 may be configured to process the first mode ultrasound image to determine a location of at least the target anatomical structure. In an exemplary embodiment, the processing of the first mode ultrasound image by the object identification processor 160 may be initiated in response to receiving a user selection to switch to a second mode, such as Color Flow, Power Doppler, B-Flow Color, or the like. In certain embodiments, the object identification processor 160 may continuously process received first mode ultrasound images to detect if the ultrasound probe 104 was moved to acquire a different standard view. Alternatively, after initial processing of the first mode ultrasound images by the object identification processor 160, subsequent processing of the first mode ultrasound images may be triggered by a detected change to the target anatomical structure, as described in more detail below.

The object identification processor 160 may include image analysis algorithms, artificial intelligence algorithms, one or more deep neural networks (e.g., a convolutional neural network) and/or may utilize any suitable form of image analysis techniques or machine learning processing functionality configured to automatically determine a location of one or more anatomical structures depicted in a first mode ultrasound image, such as a target anatomical structure associated with an identified standard view. In various embodiments, the object identification processor 160 may be provided as a deep neural network that may be made up of, for example, an input layer, an output layer, and one or more hidden layers in between the input and output layers. In an exemplary embodiment, the deep neural network may be an object detection model that identifies regions within the first mode ultrasound image having particular anatomical structures. In a representative embodiment, the deep neural network may be an object segmentation model that identifies boundaries of one or more anatomical structures on a pixel-by-pixel basis within the first mode ultrasound image. Each of the layers may be made up of a plurality of processing nodes that may be referred to as neurons. For example, the object identification processor 160 may include an input layer having a neuron for each pixel or group of pixels from the ultrasound image data. The output layer may have neurons corresponding to locations of at least one of the anatomical structures depicted in the ultrasound image data. Each neuron of each layer may perform a processing function and pass the processed ultrasound image information to one of a plurality of neurons of a downstream layer for further processing. The processing performed by the object identification processor 160 deep neural network (e.g., convolutional neural network) may identify locations of anatomical structures depicted in ultrasound image data with a high degree of probability. The object identification processor 160 may provide the identified object locations to the second mode processor 170, present the identifications of the objects (i.e., anatomical structures) at the locations on the first mode ultrasound image at the display system 134, and/or may store the identified object locations at archive 138 and/or any suitable data storage medium.

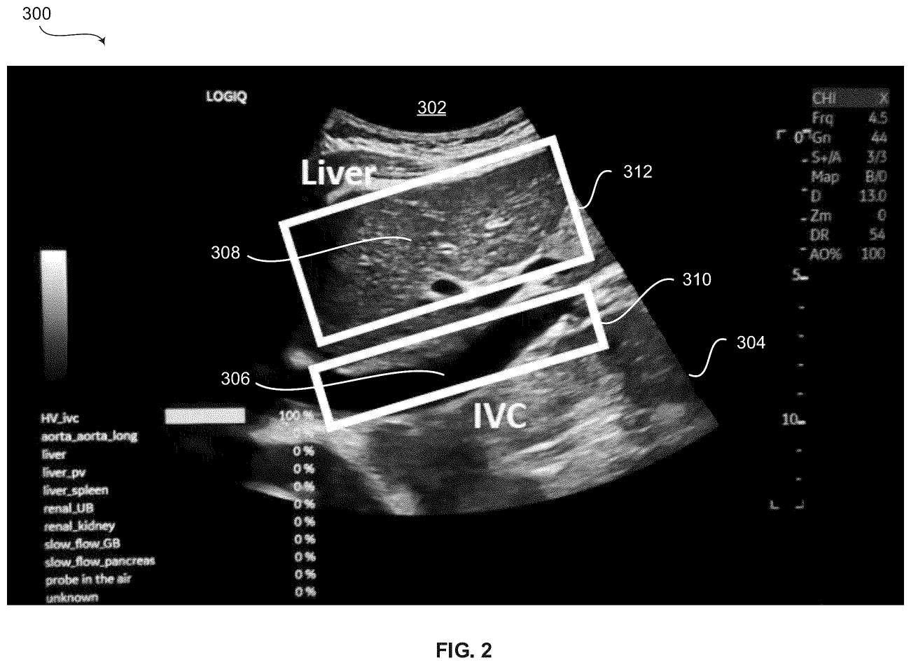

FIG. 2 illustrates a screenshot 300 of an exemplary first mode ultrasound image 304 having identified 310, 312 anatomical structures 306, 308, in accordance with various embodiments. Referring to FIG. 2, the screenshot 300 comprises an image display portion 302 having a first mode ultrasound image 304. The first mode ultrasound image 304 may be a B-mode image or any suitable image acquired according to a first mode and generated by the first mode processor 140. The first mode ultrasound image 304 may be processed by the view classification processor 150 of the signal processor 132 to classify the standard view depicted in the first mode ultrasound image 304. The standard view represented in FIG. 2 is an abdominal standard view of the inferior vena cava (IVC) 306 with a hepatic vein of a liver 308 draining into the IVC 306. In various embodiments, the view classification may be presented at the display system 134. For example, a list of standard views may be presented along with a probability each standard view is the depicted standard view. The standard view identified by the view classification processor 150 is associated with a target anatomical structure, which is the IVC 306 in the example of FIG. 2. The first mode ultrasound image 304 may be processed by the object identification processor 160 of the signal processor 132 to identify locations of at least one anatomical structure 306, 308 illustrated in the first mode ultrasound image 304. For example, the object identification processor 160 may apply an object detection model to automatically identify a first region 310 of the first mode ultrasound image 304 having the IVC 306 and a second region 312 of the first mode ultrasound image 304 having the liver 308, as shown in FIG. 2. Additionally and/or alternatively, the object identification processor 160 may apply an object segmentation model to automatically segment the anatomical structures 306, 308 illustrated in the first mode ultrasound image 304 to identify the boundaries of the anatomical structures 306, 308 in the first mode ultrasound image 304. The object detection illustrated in FIG. 2, and/or object segmentation, identifies 310, 312 the locations of the anatomical structures 306, 308, including the target anatomical structure (i.e., the IVC 306 in the example of FIG. 2), illustrated in the first mode ultrasound image 304. The first mode ultrasound image 304 having the identified regions 310, 312 of the anatomical structures 306, 308 may be provided to the second mode processor 170, presented at display system 134, and/or stored at archive 138 and/or any suitable data storage medium.

Referring again to FIG. 1, the signal processor 132 may include a second mode processor 170 that comprises suitable logic, circuitry, interfaces and/or code that may be operable to automatically select a region of interest box geometry and second mode imaging settings for acquiring second ultrasound image information according to a second mode by the ultrasound probe 104. For example, the second mode processor 170 may be configured to receive from the view classification processor 150 and object identification processor 160, or retrieve from the archive 138 or any suitable data storage medium, the target anatomical structure associated with the standard view and the location of the target anatomical structure in the first mode ultrasound image.

The second mode processor 170 may be configured to select a region of interest box geometry, which includes a location of the region of interest box in the first mode ultrasound image, a depth start for the region of interest box, a depth end for the region of interest box, a width of the region of interest box, and the like. The region of interest box geometry parameters may be dependent upon the type of the ultrasound probe 104. For example, a region of interest box width may be a distance for linear probes or an angle for curved probes. The region of interest box geometry automatically selected by the second mode processor 170 is based on the target anatomical structure associated with the standard view and the location of the target anatomical structure in the first mode ultrasound image. For example, the location of the region of interest box is centered on the target anatomical structure in the first mode ultrasound image and sized based on the size and location of the target anatomical structure in the first mode ultrasound image.

The second mode processor 170 may be configured to automatically select the second mode imaging settings based on the target anatomical structure associated with the standard view and the location of the target anatomical structure in the first mode ultrasound image, as identified by the view classification processor 150 and object identification processor 160. The second mode imaging settings may include, for example, gain, frequency, line density/frame average (L/A), pulse repetition frequency (PRF), wall filter (WF), spatial filter/packet size (S/P), acoustic output, and/or any suitable imaging settings for a second mode, such as Color Flow, Power Doppler, B-Flow Color, or the like. The selection of the second mode imaging settings is based on the first mode information (i.e., view classification and object identification) provided by the view classification processor 150 and object identification processor 160. For example, different target anatomical structures may correspond with different second mode imaging settings due to the different hemodynamics of the different anatomical structures. As another example, the location of the target anatomical structure in the first mode ultrasound image may influence the selection of various second mode imaging settings, such as a depth of the target anatomical structure in the first mode ultrasound image influencing the selected second mode frequency setting, among other things.

The ultrasound system 100 is configured to acquire second mode ultrasound information according to a second mode based on the region of interest box geometry and the second mode imaging settings automatically selected by the second mode processor 170. The second mode may be a Color Flow mode, Power Doppler mode, B-Flow Color mode, or any suitable mode. The second mode processor 170 is configured to cause the display system 134 to present the region of interest box and the acquired second mode information within the region of interest box superimposed on the first mode ultrasound image. The second mode processor 170 may be configured to update the region of interest box geometry and the second mode imaging settings in response to updated standard view classifications and/or updated anatomical object locations received from the view classification processor 150 and/or object identification processor 160, respectively. For example, the second mode processor 170 may receive an updated standard view classification and/or object location identifications if an ultrasound operator moves the ultrasound probe 104 to a different standard view to acquire ultrasound image data on a different target anatomical structure. As another example, the second mode processor 170 may receive an updated standard view classification and/or object location identifications if an ultrasound operator selects a different target anatomical structure within the same standard view, such as switching from the IVC 306 target structure to the liver 308 as a target structure in the IVC abdominal standard view shown in FIG. 2. For example, the ultrasound operator may select the different target by navigating a cursor via a user input device 130 (e.g., mousing device, trackball, etc.) over the different target anatomical structure in the displayed ultrasound image and providing a selection input (e.g., button depression), or providing a touch input of the different anatomical structure in the ultrasound image presented at a touchscreen display 130, 134. As another example, the ultrasound operator may select a different target anatomical structure from a drop down menu listing anatomical structures depicted in the current standard view. As another example, the user input device 130 may include a button to switch to a different target anatomical structure associated with a most similar standard view to the determined standard view.

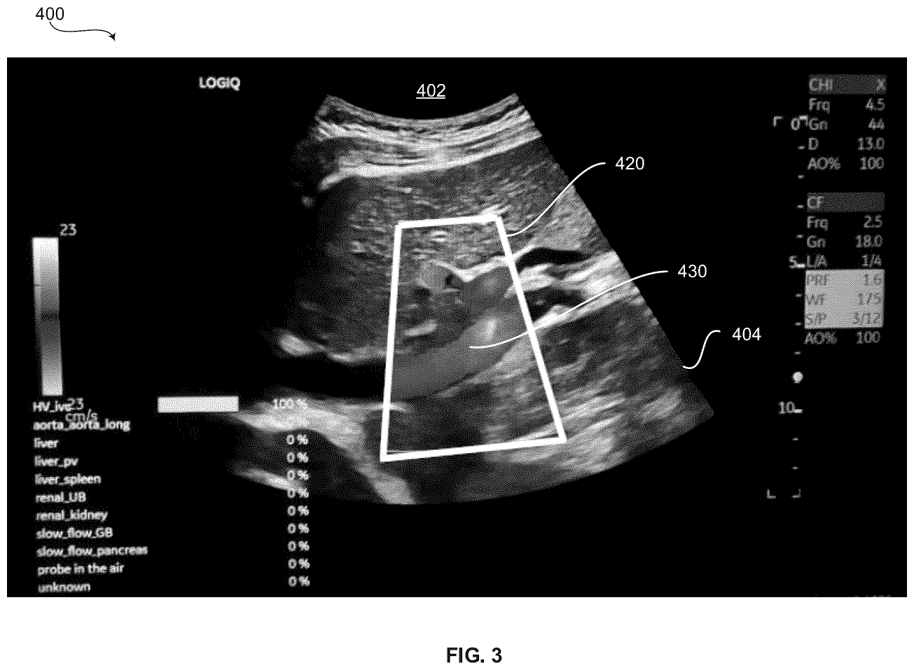

FIG. 3 illustrates a screenshot 400 of exemplary second ultrasound image information 430 within a region of interest box 420 automatically placed on a first mode ultrasound image 404, in accordance with various embodiments. Referring to FIG. 3, the screenshot 400 comprises an image display portion 402 having a first mode ultrasound image 404. The first mode ultrasound image 404 may be a B-mode image or any suitable image acquired according to a first mode and generated by the first mode processor 140, similar to the first mode ultrasound image 304 of FIG. 2. The second mode processor 170 may receive a standard view classification of the first mode ultrasound image 404 associated with a target anatomical structure from the view classification processor 150, and object locations of anatomical structures from the object identification processor 160. For example, the second mode processor 170 may receive a classification that the standard view is an abdominal standard view of the inferior vena cava (IVC) with a hepatic vein of a liver 308 draining into the IVC, which identifies the target anatomical structure as the IVC, as discussed above with reference to FIG. 2. The second mode processor 170 may receive identified regions of anatomical structures depicted in the first mode ultrasound image 404 from the object identification processor 160 inferencing an object detection model as shown in FIG. 2, or may receive segmented boundaries of anatomical structures depicted in the first mode ultrasound image 404 from the object identification processor 160 inferencing an object segmentation model. The view classification associated with a target anatomical structure and the location of the at least one anatomical structure depicted in the first mode ultrasound image defines first mode information. The second mode processor 170 is configured to automatically select a region of interest box geometry (i.e., region of interest box size and location) and automatically select second mode imaging settings based on the first mode information. The ultrasound system 100 acquires second mode ultrasound information 430 according to the second mode based on the region of interest box geometry and second mode image settings. The second mode may be a Color Flow mode as shown in FIG. 3, Power Doppler mode, B-Flow Color mode, or any suitable mode. The second mode processor 170 is configured to cause the display system 134 to present the region of interest box 420 overlaid on the first mode ultrasound image 404 with the second mode ultrasound information 430 superimposed on the first mode ultrasound image 404 within the region of interest box 420. The second ultrasound image information 430 within a region of interest box 420 automatically placed on a first mode ultrasound image 404 may be presented at display system 134 and/or stored at archive 138 and/or any suitable data storage medium.

Referring again to FIG. 1, the view classification processor 150 and object identification processor 160 may continuously process the first mode ultrasound images 304, 404 as the image data is acquired and the images 304, 404 are generated by the first mode processor 140. Accordingly, the view classification processor 150 may detect a new standard view classification if the ultrasound probe 104 is moved to a different position. The object identification processor 160 may identify new anatomical object locations 310, 312 if the ultrasound probe 104 is moved to a different position, for example. Additionally and/or alternatively, the signal processor 132 and/or the object identification processor 160 may continuously process the first mode image data within the region of interest box 420 to determine whether the target anatomical structure 306 has changed, such as due to movement of the ultrasound probe 104. In this regard, the signal processor 132 and/or object identification processor 160 may include image analysis algorithms, artificial intelligence algorithms, one or more deep neural networks (e.g., a convolutional neural network) and/or may utilize any suitable form of image analysis techniques or machine learning processing functionality configured to determine whether a target anatomical structure 306 within the region of interest box 420 has changed. The determination that the target anatomical structure 306 has changed may trigger processing of the entire first mode ultrasound image 304, 404 by the view classification processor 150 and the object identification processor 160 to determine the updated standard view and the location of the anatomical structures illustrated in the updated standard view. The optional continuous processing of only the first mode image data within the region of interest box 430 to trigger processing of the entire first mode ultrasound image 304, 404, instead of continuous processing of the entire first mode ultrasound image 304, 404, reduces use of computational resources to improve the functioning of the ultrasound system 100.

The display system 134 may be any device capable of communicating visual information to a user. For example, a display system 134 may include a liquid crystal display, a light emitting diode display, and/or any suitable display or displays. The display system 134 can be operable to present first mode ultrasound images 304, 404, identified object regions 310, 312 and/or segmented object boundaries, standard view classifications, imaging settings, a region of interest box 420, second mode ultrasound image data 430, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores first mode ultrasound images 304, 404, identified object regions 310, 312 and/or segmented object boundaries within first mode ultrasound images 304, 404, standard view classifications, imaging settings, second mode ultrasound image data 430 within a region of interest box 420 overlaid on first mode ultrasound images 304, 404, instructions for classifying standard views, instructions for identifying object regions, instructions for identifying object boundaries, instructions for selecting a region of interest box geometry, and/or instructions for selecting second mode imaging settings, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 210 may comprise suitable logic, circuitry, interfaces and/or code that may be operable to train the neurons of the deep neural networks (e.g., artificial intelligence model(s)) inferenced (i.e., deployed) by the signal processor 132, view classification processor 150, and/or object identification processor 160. For example, the training engine 210 may apply classified anatomical structures to train the view classification, object detection, and/or object segmentation networks inferenced by the view classification processor 150, and/or object identification processor 160 to automatically classify standard views, automatically detect regions 310, 312 having anatomical structures 306, 308, and/or automatically segment anatomical structures 306, 308 in first mode ultrasound images 304, 404. The classified anatomical structures may include an input image and a ground truth binary image (i.e., mask) of the manually classified standard views, manually detected object regions 310, 312, and/or manually segmented anatomical structures 306, 308. The training engine 210 may be configured to optimize the view classification, object detection, and/or object segmentation networks by adjusting the weighting of the view classification, object detection, and/or object segmentation networks to minimize a loss function between the input ground truth mask and an output predicted mask.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms.

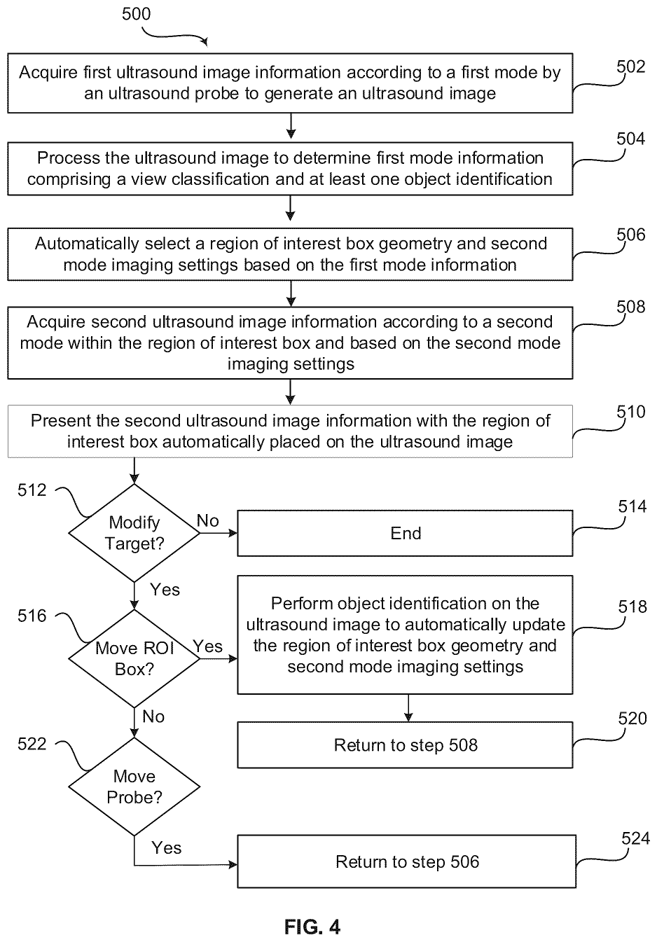

FIG. 4 is a flow chart 500 illustrating exemplary steps 502-524 that may be utilized for automatically selecting imaging settings and automatically placing a region of interest box 420 on a first mode ultrasound image 304, 404 based on first mode ultrasound image information 310, 312 when entering a second ultrasound imaging mode, in accordance with various embodiments. Referring to FIG. 4, there is shown a flow chart 500 comprising exemplary steps 502 through 524. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 502, an ultrasound probe 104 of an ultrasound system 100 may acquire a first ultrasound image information according to a first mode to generate a first mode ultrasound image 304, 404. For example, an ultrasound probe 104 in the ultrasound system 100 may be operable to perform an ultrasound scan of a region of interest, such as an abdominal region. The ultrasound scan may be performed according to the first mode, such as a B-mode or any suitable image acquisition mode. The first ultrasound image dataset may be received by the first mode processor 140 of the signal processor 132 and/or stored to archive 138 or any suitable data storage medium from which the first mode processor 140 may retrieve the first ultrasound image information. The first mode processor 140 of the signal processor 132 of the ultrasound system 100 may be configured to process the acquired and/or retrieved first mode ultrasound image information to generate ultrasound images according to the first mode. As an example, the first mode may be a B-mode and the first mode processor 140 may be configured to process received first mode ultrasound image information into B-mode image(s) 304, 404.

At step 504, the signal processor 132 of the ultrasound system 100 may process the first mode ultrasound image 304, 404 to determine first mode information comprising a view classification and at least one object identification. For example, a view classification processor 150 of the signal processor 132 of the ultrasound system 100 may be configured to automatically determine a standard view depicted in a first mode ultrasound image 304, 404, such as an abdomen standard view depicted in a B-mode image. In various embodiments, the standard view is associated with a target anatomical structure. Accordingly, determining the standard view by the view classification processor 150 identifies a target anatomical structure. In a representative embodiment, the view classification processor 150 inferences a view classification deep learning model, or any suitable image analysis algorithms, to classify the standard view depicted in the first mode ultrasound image 304, 404. An object identification processor 160 of the signal processor 132 of the ultrasound system 100 may be configured to process the first mode ultrasound image 304, 404 generated by the first mode processor 140 at step 502 to determine anatomical structure locations 310, 312 depicted in the first mode ultrasound image 304, 404. The object identification processor 160 may determine locations of, for example, one or more of a liver, kidney, gallbladder, aorta, pancreas, spleen, and/or inferior vena cava, among other anatomical structures, depicted in a first mode ultrasound image 304, 404 that is an abdominal standard view. In various embodiments, the object identification processor 160 may receive, from the view classification processor 150, the standard view classification, which is associated with a target anatomical structure 306. Accordingly, the object identification processor 160 may be configured to process the first mode ultrasound image 304, 404 to determine a location 310 of at least the target anatomical structure 306. In a representative embodiment, the object identification processor 160 inferences an object detection deep learning model, an object segmentation deep learning model, or any suitable image analysis algorithms, to identify regions 310, 312 or segmented boundaries of at least one anatomical structure 306, 308 depicted in the first mode ultrasound image 304, 404. In an exemplary embodiment, the processing of the first mode ultrasound image 304, 404 by the view classification processor 150 and/or the object identification processor 160 may be initiated in response to receiving a user selection to switch to a second mode, such as Color Flow, Power Doppler, B-Flow Color, or the like.

At step 506, the signal processor 132 of the ultrasound system 100 may automatically select a region of interest box geometry and second mode imaging settings based on the first mode information determined at step 504. For example, a second mode processor 170 of the signal processor 132 may be configured to receive from the view classification processor 150 and object identification processor 160, or retrieve from the archive 138 or any suitable data storage medium, the target anatomical structure 306 associated with the standard view and the location of the target anatomical structure 310 in the first mode ultrasound image 304, 404. The second mode processor 170 may be configured to select a region of interest box geometry, which includes a location of the region of interest box 420 in the first mode ultrasound image 304, 404, a depth start for the region of interest box, a depth end for the region of interest box 420, a width of the region of interest box 420, and the like. The region of interest box geometry automatically selected by the second mode processor 170 is based on the target anatomical structure 306 associated with the standard view and the location 310 of the target anatomical structure 306 in the first mode ultrasound image 304, 404. For example, the location of the region of interest box 420 is centered on the target anatomical structure 306 in the first mode ultrasound image 304, 404 and sized based on the size and location of the target anatomical structure 306 in the first mode ultrasound image 304, 404. The second mode processor 170 may be configured to automatically select the second mode imaging settings based on the target anatomical structure 306 associated with the standard view and the location 310 of the target anatomical structure 306 in the first mode ultrasound image 304, 404, as identified by the view classification processor 150 and object identification processor 160. The second mode imaging settings may include, for example, gain, frequency, line density/frame average (L/A), pulse repetition frequency (PRF), wall filter (WF), spatial filter/packet size (S/P), acoustic output, and/or any suitable imaging settings for a second mode, such as Color Flow, Power Doppler, B-Flow Color, or the like. The selection of the second mode imaging settings is based on the first mode information (i.e., view classification and object identification) provided by the view classification processor 150 and object identification processor 160.

At step 508, the ultrasound probe 104 of the ultrasound system 100 may acquire second ultrasound image information 430 according to a second mode within the region of interest box 420 and based on the second mode imaging settings. For example, the ultrasound probe 104 of the ultrasound system 100 may acquire second mode ultrasound information according to a second mode based on the region of interest box geometry and the second mode imaging settings automatically selected by the second mode processor 170 at step 506. The second mode may be a Color Flow mode, Power Doppler mode, B-Flow Color mode, or any suitable mode.

At step 510, the signal processor 132 may cause a display system 134 of the ultrasound system 100 to present the second ultrasound image information 430 with the region of interest box 420 automatically placed on the first mode ultrasound image 304, 404. For example, the second mode processor 170 is configured to cause the display system 134 to present the region of interest box 420 and the acquired second ultrasound image information 430 within the region of interest box 420 superimposed on the first mode ultrasound image 304, 404.

At step 512, the signal processor 132 of the ultrasound system 100 may determine whether the target anatomical structure 306 has been modified. If second mode ultrasound information 430 has been acquired for all desired target anatomical structures, the process proceeds to step 514. If an ultrasound operator desires to acquire second mode ultrasound information 430 for additional target anatomical structures, the ultrasound operator may select a new target anatomical structure within the same standard view (i.e., without moving the ultrasound probe 104) at step 516 or may manipulate the ultrasound probe 104 to a different position to acquire a different standard view associated with a new target anatomical structure at step 522.

At step 514, the process 500 ends when second mode ultrasound information has been acquired for all of the desired target anatomical structures.

At step 516, the ultrasound operator may select a new target anatomical structure within the same standard view (i.e., without moving the ultrasound probe 104). For example, the ultrasound operator may select a different target anatomical structure within the same standard view, such as switching from the IVC 306 target structure to the liver 308 as a target structure in the IVC abdominal standard view shown in FIG. 2. As an example, the ultrasound operator may select the different target by navigating a cursor via a user input device 130 (e.g., mousing device, trackball, etc.) over the different target anatomical structure in the displayed ultrasound image and providing a selection input (e.g., button depression), or providing a touch input of the different anatomical structure in the ultrasound image presented at a touchscreen display 130, 134. As another example, the ultrasound operator may select a different target anatomical structure from a drop down menu listing anatomical structures depicted in the current standard view. As another example, the user input device 130 may include a button to switch to a different target anatomical structure associated with a most similar standard view to the determined standard view.

At step 518, the signal processor 132 of the ultrasound system 100 performs object identification on the first mode ultrasound image 304, 404 to automatically update the region of interest box geometry and the second mode imaging settings. For example, the object identification processor 160 of the signal processor 132 may deploy an object detection deep learning model, an object segmentation deep learning model, or any suitable image analysis algorithms to identify the location (e.g., region or segmented boundary) of the new target anatomical structure. The second mode processor 170 may be configured to update the region of interest box geometry and the second mode imaging settings in response to modified target anatomical structure and the updated anatomical object locations received from the object identification processor 160.

At step 520, the process returns to step 508 to acquire second ultrasound image information 430 according to the second mode within the region of interest box 420 and based on the second mode imaging settings updated at step 518 in response to the new target anatomical structure.

At step 522, the ultrasound operator may manipulate the ultrasound probe 104 to a different position to acquire a different standard view associated with a new target anatomical structure. In various embodiments, the view classification processor 150 and object identification processor 160 may continuously process the first mode ultrasound images 304, 404 as the image data is acquired and the images 304, 404 are generated by the first mode processor 140. Accordingly, the view classification processor 150 may detect a new standard view classification if the ultrasound probe 104 is moved to a different position. The object identification processor 160 may identify new anatomical object locations 310, 312 if the ultrasound probe 104 is moved to a different position, for example. Additionally and/or alternatively, the signal processor 132 and/or the object identification processor 160 may continuously process the first mode image data within the region of interest box 420 to determine whether the target anatomical structure 306 has changed, such as due to movement of the ultrasound probe 104. In this regard, the signal processor 132 and/or object identification processor 160 may include image analysis algorithms, artificial intelligence algorithms, one or more deep neural networks (e.g., a convolutional neural network) and/or may utilize any suitable form of image analysis techniques or machine learning processing functionality configured to determine whether a target anatomical structure 306 within the region of interest box 420 has changed. The determination that the target anatomical structure 306 has changed may trigger processing of the entire first mode ultrasound image 304, 404 by the view classification processor 150 and the object identification processor 160 to determine the updated standard view and the location of the anatomical structures illustrated in the updated standard view.

At step 524, the process returns to step 506 to automatically select a region of interest box geometry and second mode imaging settings based on the first mode information determined at step 522 in response to the ultrasound probe movement to acquire a different standard view of a new target anatomical structure.

Aspects of the present disclosure provide a method 500 and system 100 for automatically selecting imaging settings and automatically placing a region of interest box 420 on a first mode ultrasound image (e.g., B-mode image) 304, 404 based on first mode ultrasound image information when entering a second ultrasound imaging mode (e.g., Color Flow, Power Doppler, B-Flow Color, or the like). In accordance with various embodiments, the method 500 may comprise acquiring 502, by an ultrasound probe 104 of an ultrasound system 100, first ultrasound image information according to a first mode. The first ultrasound image information comprises a first mode ultrasound image 304, 404. The method 500 may comprise processing 504, by at least one processor 132, 150, 160 of the ultrasound system 100, the first mode ultrasound image 304, 404 to determine first mode information. The method 500 may comprise automatically selecting 506, by the at least one processor 132, 170, a size and a location of a region of interest box 420 based on the first mode information. The method 500 may comprise acquiring 508, by the ultrasound probe 104, second ultrasound image information 430 according to a second mode based on the region of interest box 420. The method 500 may comprise causing 510, by the at least one processor 132, 170, the display system 134 to present the second ultrasound image information 430 and the region of interest box 420 automatically placed on the first mode ultrasound image 304, 404.

In an exemplary embodiment, the first mode is B-mode and the second mode is one of a Color Flow mode, Power Doppler mode, or B-Flow Color mode. In a representative embodiment, the first mode information comprises an ultrasound standard view classification and at least one anatomical object identification 310, 312. In various embodiments, the method 500 comprises inferencing 504, by the at least one processor 132, 150, an ultrasound view classification model to determine the ultrasound standard view classification. In certain embodiments, the method 500 comprises inferencing 504, by the at least one processor 132, 160, an object detection model or an object segmentation model to determine the at least one anatomical object identification 310, 312. In an exemplary embodiment, the ultrasound standard view classification is associated with a target anatomical object 306. The at least one anatomical object identification defines a location 310 of the target anatomical object 306. In a representative embodiment, the method comprises automatically selecting 506, by the at least one processor 132, 170, second mode imaging settings based on the first mode information. The second ultrasound image information 430 is acquired based on the second mode imaging settings.

Various embodiments provide a system 100 for automatically selecting imaging settings and automatically placing a region of interest box 420 on a first mode ultrasound image (e.g., B-mode image) 304, 404 based on first mode ultrasound image information when entering a second ultrasound imaging mode (e.g., Color Flow, Power Doppler, B-Flow Color, or the like). The ultrasound system 100 may comprise an ultrasound probe 104, a display system 134, and at least one processor 132, 140, 150, 160, 170. The ultrasound probe 104 may be operable to acquire first ultrasound image information according to a first mode. The first ultrasound image information comprises a first mode ultrasound image 304, 404. The ultrasound probe 104 may be operable to acquire second ultrasound image information 430 according to a second mode based on a region of interest box 420. The at least one processor 132, 150, 160 may be configured to process the first mode ultrasound image 304, 404 to determine first mode information. The at least one processor 132, 170 may be configured to automatically select a size and a location of the region of interest box 420 based on the first mode information. The display system 134 may be configured to present the second ultrasound image information 430 and the region of interest box 420 automatically placed on the first mode ultrasound image 304, 404.

In a representative embodiment, the first mode is B-mode, and the second mode is one of a Color Flow mode, Power Doppler mode, or B-Flow Color mode. In various embodiments, the first mode information comprises an ultrasound standard view classification and at least one anatomical object identification 310, 312. In certain embodiments, the ultrasound standard view classification is determined based on inferencing an ultrasound view classification model. In an exemplary embodiment, the at least one anatomical object identification 310, 312 is based on inferencing an object detection model or an object segmentation model. In a representative embodiment, the ultrasound standard view classification is associated with a target anatomical object 306. The at least one anatomical object identification defines a location 310 of the target anatomical object 306. In various embodiments, the second ultrasound image information 430 is acquired based on second mode imaging settings. The at least one processor 132, 170 is configured to automatically select the second mode imaging settings based on the first mode information.

Certain embodiments provide a system 100 for automatically selecting imaging settings and automatically placing a region of interest box 420 on a first mode ultrasound image (e.g., B-mode image) 304, 404 based on first mode ultrasound image information when entering a second ultrasound imaging mode (e.g., Color Flow, Power Doppler, B-Flow Color, or the like). The ultrasound system 100 may comprise an ultrasound probe 104, a display system 134, and at least one processor 132, 140, 150, 160, 170. The ultrasound probe 104 may be operable to acquire first ultrasound image information according to a first mode. The first ultrasound image information comprises a first mode ultrasound image 304, 404. The ultrasound probe 104 may be operable to acquire second ultrasound image information 430 according to a second mode based on a region of interest box 420. The at least one processor 132, 150, 160 may be configured to process the first mode ultrasound image 304, 404 to determine first mode information. The at least one processor 132, 170 may be configured to cause a display system 134 to present the second ultrasound image information 430 with a region of interest box 420 automatically placed over a first target anatomical object 306 in the first mode ultrasound image 304, 404 based on the first mode information. The at least one processor 132, 150 may be configured to change the first target anatomical object 306 to a second target anatomical object. The at least one processor 132, 170 may be configured to automatically adjust a size and a location of the region of interest box 420 placed over the second target anatomical object in the first mode ultrasound image 304, 404 based on the first mode information and in response to the change of the first target anatomical object 306 to the second target anatomical object. The display system 134 may be configured to present the second ultrasound image information 430 and the region of interest box 420 automatically placed on the first mode ultrasound image 304, 404.

In various embodiments, the first mode information comprises an ultrasound standard view classification determined by inferencing an ultrasound view classification model, and at least one anatomical object identification 310, 312 determined by inferencing an object detection model or an object segmentation model. In certain embodiments, the second ultrasound image information 430 is acquired based on second mode imaging settings. The at least one processor 132, 170 is configured to automatically select the second mode imaging settings based on the first mode information. In an exemplary embodiment, the at least one processor 132, 150, 160 is configured to continuously process an entirety of the first mode ultrasound image 304, 404 to detect movement of the ultrasound probe 104, and determine updated first mode information based on the movement of the ultrasound probe 104. The updated first mode information comprises the change of the first target anatomical object 306 to the second target anatomical object. In a representative embodiment, the at least one processor 132, 160 is configured to continuously process a portion of the first mode ultrasound image 304, 404 within the region of interest box 420 to determine the first target anatomical object 306 has moved out of the region of interest box 420 due to movement of the ultrasound probe 104. The at least one processor 132, 150, 160 is configured to process an entirety of the first mode ultrasound image 304, 404 to determine updated first mode information in response to determining that the first target anatomical object 306 has moved out of the region of interest box 420 due to the movement of the ultrasound probe 104. The updated first mode information comprises the change of the first target anatomical object 306 to the second target anatomical object. In various embodiments, the at least one processor 132, 150, 170 is configured to change the first target anatomical object 306 to the second target anatomical object in response to a user selection of the second target anatomical object in the first mode ultrasound image 304, 404.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for automatically selecting imaging settings and automatically placing a region of interest box on a first mode ultrasound image (e.g., B-mode image) based on first mode ultrasound image information when entering a second ultrasound imaging mode (e.g., Color Flow, Power Doppler, B-Flow Color, or the like).

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method, comprising:
acquiring, by an ultrasound probe of an ultrasound system, first ultrasound image information according to a first mode, wherein the first ultrasound image information comprises a first mode ultrasound image;
processing, by at least one processor of the ultrasound system, the first mode ultrasound image to determine first mode information;
automatically selecting, by the at least one processor, a size and a location of a region of interest box based on the first mode information;
acquiring, by the ultrasound probe, second ultrasound image information according to a second mode based on the region of interest box; and
causing, by the at least one processor, a display system to present the second ultrasound image information and the region of interest box automatically placed on the first mode ultrasound image.

2. The method of claim 1, wherein:
the first mode is B-mode; and
the second mode is one of a Color Flow mode, Power Doppler mode, or B-Flow Color mode.

3. The method of claim 1, wherein the first mode information comprises an ultrasound standard view classification and at least one anatomical object identification.

4. The method of claim 3, comprising inferencing, by the at least one processor, an ultrasound view classification model to determine the ultrasound standard view classification.

5. The method of claim 3, comprising inferencing, by the at least one processor, an object detection model or an object segmentation model to determine the at least one anatomical object identification.

6. The method of claim 3, wherein:
the ultrasound standard view classification is associated with a target anatomical object; and
the at least one anatomical object identification defines a location of the target anatomical object.

7. The method of claim 1, comprising automatically selecting, by the at least one processor, second mode imaging settings based on the first mode information, wherein the second ultrasound image information is acquired based on the second mode imaging settings.

8. An ultrasound system, comprising:
an ultrasound probe operable to:
acquire first ultrasound image information according to a first mode, wherein the first ultrasound image information comprises a first mode ultrasound image; and
acquire second ultrasound image information according to a second mode based on a region of interest box;
at least one processor configured to:
process the first mode ultrasound image to determine first mode information; and
automatically select a size and a location of the region of interest box based on the first mode information; and
a display system configured to present the second ultrasound image information and the region of interest box automatically placed on the first mode ultrasound image.

9. The system of claim 8, wherein:
the first mode is B-mode; and
the second mode is one of a Color Flow mode, Power Doppler mode, or B-Flow Color mode.

10. The system of claim 8, wherein the first mode information comprises an ultrasound standard view classification and at least one anatomical object identification.

11. The system of claim 10, wherein the ultrasound standard view classification is determined based on inferencing an ultrasound view classification model.

12. The system of claim 10, wherein the at least one anatomical object identification is based on inferencing an object detection model or an object segmentation model.

13. The system of claim 10, wherein:
the ultrasound standard view classification is associated with a target anatomical object; and
the at least one anatomical object identification defines a location of the target anatomical object.

14. The system of claim 8, wherein:
the second ultrasound image information is acquired based on second mode imaging settings; and
the at least one processor is configured to automatically select the second mode imaging settings based on the first mode information.

15. An ultrasound system, comprising:
an ultrasound probe operable to:
acquire first ultrasound image information according to a first mode, wherein the first ultrasound image information comprises a first mode ultrasound image; and
acquire second ultrasound image information according to a second mode based on a region of interest box;
at least one processor configured to:
process the first mode ultrasound image to determine first mode information;
cause a display system to present the second ultrasound image information with a region of interest box automatically placed over a first target anatomical object in the first mode ultrasound image based on the first mode information;
change the first target anatomical object to a second target anatomical object; and
automatically adjusting a size and a location of the region of interest box placed over the second target anatomical object in the first mode ultrasound image based on the first mode information and in response to the change of the first target anatomical object to the second target anatomical object; and
the display system configured to present the second ultrasound image information and the region of interest box automatically placed on the first mode ultrasound image.

16. The system of claim 15, wherein the first mode information comprises:
an ultrasound standard view classification determined by inferencing an ultrasound view classification model; and
at least one anatomical object identification determined by inferencing an object detection model or an object segmentation model.

17. The system of claim 15, wherein:
the second ultrasound image information is acquired based on second mode imaging settings; and
the at least one processor is configured to automatically select the second mode imaging settings based on the first mode information.

18. The system of claim 15, wherein the at least one processor is configured to continuously process an entirety of the first mode ultrasound image to:
detect movement of the ultrasound probe; and
determine updated first mode information based on the movement of the ultrasound probe, wherein the updated first mode information comprises the change of the first target anatomical object to the second target anatomical object.

19. The system of claim 15, wherein:
the at least one processor is configured to continuously process a portion of the first mode ultrasound image within the region of interest box to determine the first target anatomical object has moved out of the region of interest box due to movement of the ultrasound probe; and
the at least one processor is configured to process an entirety of the first mode ultrasound image to determine updated first mode information in response to determining that the first target anatomical object has moved out of the region of interest box due to the movement of the ultrasound probe, wherein the updated first mode information comprises the change of the first target anatomical object to the second target anatomical object.

20. The system of claim 15, wherein the at least one processor is configured to change the first target anatomical object to the second target anatomical object in response to a user selection of the second target anatomical object in the first mode ultrasound image.
